(19) United States
(12) Patent Application Publication
(10) Pub. No.: US 2026/0344630 A1
(43) Pub. Date: Nov. 5, 2026

(54) All-Weather Butterfly Habitat

(71) Applicant: Brooksany Jean Jewell, Brooklyn, NY (US)

(72) Inventor: Brooksany Jean Jewell, Brooklyn, NY (US)

(21) Appl. No.: 19/196,148

(22) Filed: May 1, 2025

(51) Int. Cl. A01K 67/30

(52) U.S. Cl. CPC A01K 67/30

(57) ABSTRACT

The All-Weather Butterfly Habitat allows for the safe rearing of both migratory and overwintering species of butterflies from egg to adult. It solves for a variety of issues including but not limited to: protection from predation, shelter from harsh weather, fresh host plant material, and the safe and easy release of adult butterflies. The fully enclosed, A-line structure protects eggs and caterpillars from predators and won't collapse under heavy rain and snow accumulation that happens with other types of commonly used habitats with flat tops. It provides a host-plant growing area that can easily be replenished throughout the season via the detachable bottom or front zipper opening. A top flap opens for “no-touch” release. It is made of breathable material that allows in light and can be kept outside all year in most climates. It creates a protected, natural space for the duration of a butterfly's life cycle.

### CROSS REFERENCE TO RELATED APPLICATION

No prior patent application has been submitted

### STATEMENT REGARDING FEDERALLY SPONSORED RESEARCH OR ADDITIONAL RESEARCH PARTIES

Not Applicable

### REFERENCE TO A SEQUENCE LISTING

Not Applicable

### BACKGROUND OF THE INVENTION

Having reared many types of butterflies over a 15-year period. I noticed multiple issues and problems with commercially available habitats, notably poor construction meant only for fair weather “viewing” and not for the proper rearing of the insects' through all aspects of the lifecycle. So, I built my own.

### BRIEF SUMMARY OF THE INVENTION

A durable, all-weather habitat for the safe, natural rearing of both migratory and overwintering butterfly species through the entire lifecycle from egg to adult.

### BRIEF DESCRIPTION OF THE SEVERAL VIEWS OF THE DRAWINGS

FIG. 1: Is a front perspective view of the habitat, with the screen in use.

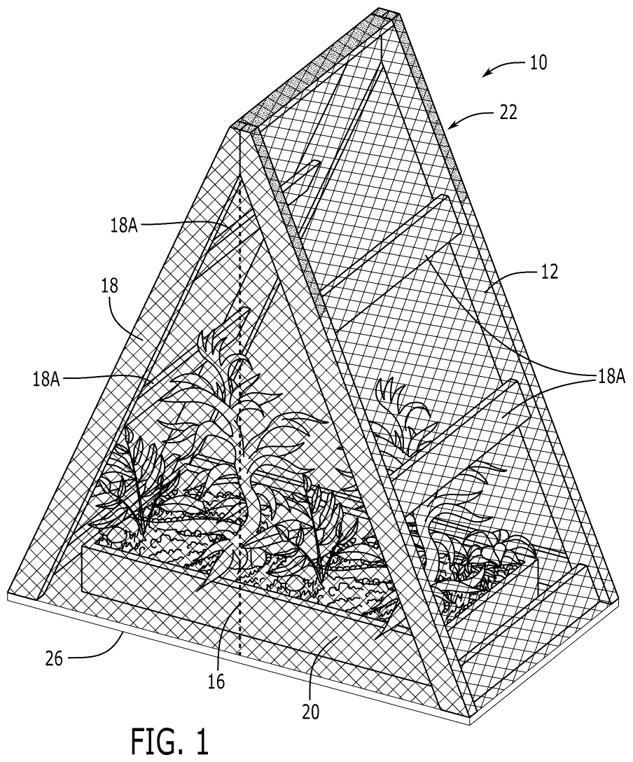

FIG. 2: Is a front perspective of the frame structure with the removable planter in use.

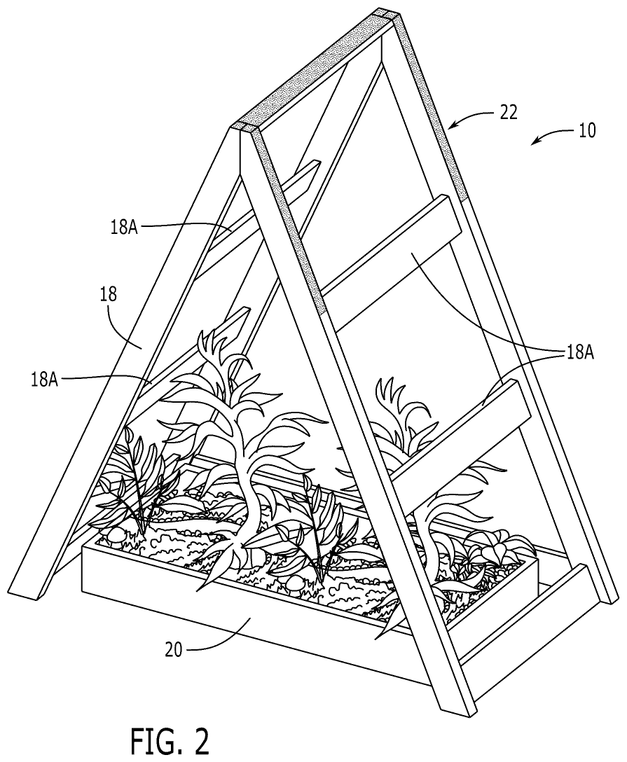

FIG. 3: Is a side perspective with the side flap open to allow for “touchless” release of adult butterflies.

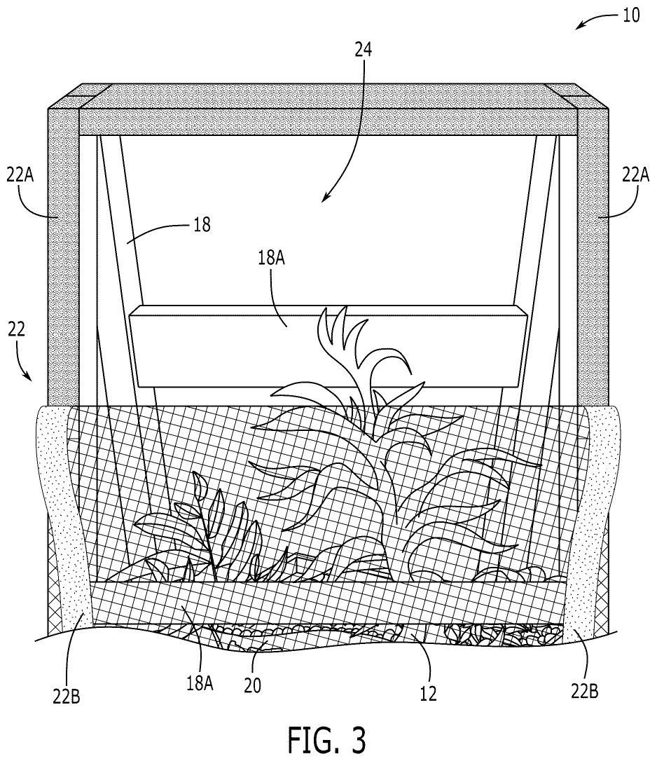

FIG. 4: is a front perspective view, broken away, showing the protected bottom layer of the habitat.

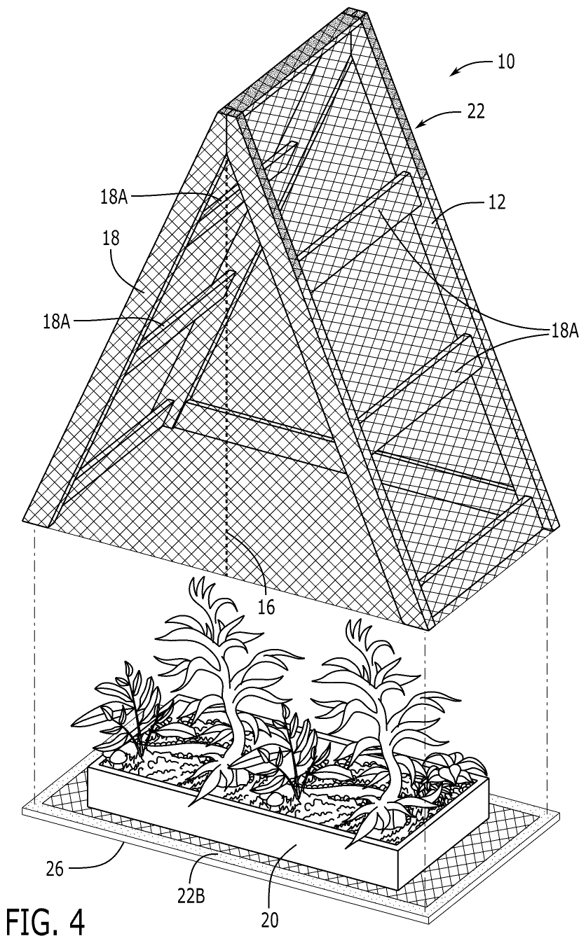

### DETAILED DESCRIPTION OF THE INVENTION

How Does It Work: This habitat allows for the safe rearing of all types of butterflies both migratory (such as Monarchs) and overwintering species (such as Swallowtails). The fully enclosed, A-line structure protects eggs and caterpillars from predators and won't collapse under harsh conditions such as rain and snow accumulation that happens with other types of commonly used, flat-top habitats. It also provides a host plant growing and seeding area that can easily be replenished during the summer months through the detachable bottom or front zipper opening. It has a top flap for no-touch release, and it is breathable, allows in light, and can be kept outside all year round in most climates in the US. It also offers areas (wood and plant material) for overwintering butterflies to create chrysalises in a protected environment. Those chrysalises will then hatch and be ready for release the following spring, creating a natural space for the duration of a butterfly's life cycle.
What problems does it solve: Commercial mesh and plastic habitats currently on the market have flat or sloped tops that collapse or tear under the weight of rain or snow and the plastic walls and/or viewing panels crack easily in cold weather. Thus, they don't properly protect the butterfly pupa—especially those that need to be kept overwinter in cold climates (like Swallowtails). This means that they are only good for summer use and are generally flimsy and not good for multiple seasons. The plastic sheeting or globe style habitats restrict air movement so they don't provide a natural outdoor environment for migratory butterflies. Most also do not allow for the direct growing of host plants within the habitat to rear insects from egg to chrysalis naturally in one place. They also don't provide for easy access to replace plants, add insects, or easily and safely capture and release adult butterflies. Other types of enclosures, such as plant protectors or animal cages leave gaps around the doors/window frames or have open bottoms that allow entry points for smaller, natural predators such as spiders, wasps, slugs, etc.
How does the invention solve these problems: My habitat is a sturdy, A-line shaped structure, which prevents collapse and tearing from the accumulation of heavy snow and rain, which occurs with a squared or flat-topped habitat. My habitat is completely enclosed in fine black garden netting that not only provides easy visibility to the insects and subtly shades caterpillars in direct sunlight but also offers superb protection from all types of natural predators from parasitic wasps to birds. It also has no plastic sheeting or viewing panels, which can crack or tear in colder temperatures, thus allowing for year after year, multi-season use. It also simulates a purely natural outdoor environment. Studies have shown that Monarchs in captivity are less likely to migrate. The hope is that a simulated but protected natural outdoor enclosure will encourage natural migration instincts. It is also built with a sturdy wooden frame that can be folded (similar to a ladder) when not in use. It features a foldable cloth-based garden insert in which host plants are grown to support the development from egg, to caterpillar, to chrysalis all in one area. A zippered front allows easy access to add insects (at any stage), as well as tend to (prune, replace, add) host plants as needed. The netted top structure of my habitat is connected to the base by hook and eye strips fixed to the netting to create a strong seal but also allow for the top portion to be detached from the base to allow for easy access to host plants and to clean the habitat in between rearing seasons. Finally, there is a hook and eye flap at the top of the A-frame structure that can be opened to also allow for easy, no-touch release of adult butterflies to eliminate the possibility of injuring them.
How is the invention an improvement: The habitat provides a natural, well-protected, predator-resistant, mesh-only (no breakable plastic) all-weather solution to rearing both migratory and over-wintering butterfly species.

AMENDED: Relationship between components:

The habitat (10) features a unique A-line structure (18), with supporting panels (18A) that must be completely encased in the fine mesh netting (12), without plastic tops or viewing panels to fully protect developing caterpillars and pupa from predators, and to avoid breakage or collapse due to cold, snow or heavy rains. The net base (26) must be fully detachable from the upper structure to allow for easy additions of new plants and cleaning between seasons. The planter insert (20) allows for direct planting and re-planting of host plants for feeding caterpillars. The planter insert should be filled with potting soil and seeds or host plants for the types of butterflies being raised. The habitat has a zippered front (16) to allow for the addition of caterpillars or eggs and the capture and release of adult butterflies. The habitat also features a detachable flap opened via hook and eye fasteners (22A and 22B) to allow for no-touch release of adult butterflies.

To build: The current structure is 42 inches high by 39 inches long, and 15 inches wide, but could be made in varying sizes/widths to adapt to the needs of the person rearing the butterflies.

The A-line wooden structure is comprised of two panels hinged at the top. The panels are each comprised of two side pieces of wood 42 inches long, connected and hinged at the top as mentioned. They are supported across the width by four 15-inch support panels spaced roughly 10.5 inches apart. The habitat panels are reinforced at the bottom (front and back) by two 39-inch boards that are hinged in the middle to allow for the structure to be folded (similar to a ladder mechanism). Structure should be covered on all sides with mesh netting that is connected to all key points of the structure, and with a zippered opening in the front to allow access, as well as a flap at the top right sealed with hook and loop strips. The upper A-line structure should be fitted with a detachable base made of netting that adheres approximately four inches up on all sides of the upper structure and attached to the upper structure netting with hoop and loop strips to provide a seamless and secure seal to prevent insect predators from entering the habitat.

Other Potential Uses: The invention could also be potentially used to house other small outdoor animals such as birds (with the introduction of perches) or it could be used as a growing habitat for plants since it would be protecting the plants from harmful bugs or deer (however, the plants would need to be decorative since pollination wouldn't be possible).

Declaration: I, Brooksany Jean Jewell (Ne: Steed), declare that I have worked with a patent attorney to execute a patent search, and to the best of my knowledge my invention is unique and a superior improvement on existing insect habitats currently on the market.

1. An all-weather butterfly habitat comprising: a durable enclosure constructed to withstand various weather conditions and configured for the safe and natural rearing of both migratory and overwintering butterfly species through their entire lifecycle from egg to adult.
The all-weather butterfly habitat of claim 1 further comprises a detachable base screen, so the upper portion can be removed for easy cleaning in between seasons or to restock food sources.
The all-weather butterfly habitat of claim 1 is completely encased in mesh via hook and loop material and does not contain plastic sheeting on the top or sides, which is prone to crack in cold weather.

2. The all-weather butterfly habitat of claim 1, further comprises a unique a-frame structure consisting of straight longitudinal panels of equal length; joined at their top ends to form an apex and supported by horizontal, equally spaced support structures, preventing rain and snow accumulation on the habitat top.

3. The all-weather butterfly habitat of claim 1, includes a removable planter so food sources for the butterflies can be directly grown and maintained in the habitat throughout the rearing season.

4. The all-weather butterfly habitat of claim 1, also has various openings including a zippered front that allows for easy access to the enclosure to add new caterpillars/eggs as well as a side-flap for no-touch release of adult butterflies.
